SEQUENCE LISTING (19 sequences)

SEQ ID NO: 1 - DNA, 4245 bp, Artificial Sequence
  Sequence of plasmid pACYC184
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctttaatgcggtagtttatcacagttaaattgctaacgcagtcaggcaccgtgtatgaaatctaacaatgcgctcatcgtcatcctcggcaccgtcaccctggatgctgtaggcataggcttggttatgccggtactgccgggcctcttgcgggatatcgtccattccgacagcatcgccagtcactatggcgtgctgctagcgctatatgcgttgatgcaatttctatgcgcacccgttctcggagcactgtccgaccgctttggccgccgcccagtcctgctcgcttcgctacttggagccactatcgactacgcgatcatggcgaccacacccgtcctgtggatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 2 - DNA, 30 bp, Artificial Sequence
  PCR Primer
ccggagaattccggatgagcattcatcagg

SEQ ID NO: 3 - DNA, 35 bp, Artificial Sequence
  PCR Primer
cccgaaagcttatcgatgataagctgtcaaacatg

SEQ ID NO: 4 - DNA, 1529 bp, Artificial Sequence
  Artificial sequence of mutated p15A ori
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcatttacgccaaaagttggcccagggcttcccggtatcaacagggacaccaggatttatttattctgcgaagtgatcttccgccacaggtatttattcggcgcaaagtgcgtcgggtgatgctgccaacttactgatttagtgtatgatggtgtttttgaggtgctccagtggcttctgtttctatcggctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggaggaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcatcccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccacaggtaattgatttagaggagttagtcttgaagtcatgcgccggttaaggctaaactgaaaggacaagttttggtgactgcgctcctccaagccagttacctcggttcaaagagttggtagctcagagaaccttcgaaacaccgccctgcaaggcggttttttcgttctcagggcaagagattacgcgcggaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctt

SEQ ID NO: 5 - DNA, 1529 bp, Artificial Sequence
  Artificial sequence of p15A ori
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagctt

SEQ ID NO: 6 - DNA, 86 bp, Artificial Sequence
  Synthetic oligonucleotide use to construct a plasmid
tcgtgcgctctcctgttcctgcctttcggtttaccggtgtcattccgctgttatggccgcgtttgtctcatcccacgcctgacact

SEQ ID NO: 7 - DNA, 87 bp, Artificial Sequence
  Synthetic oligonucleotide use to construct a plasmid
aactgagtgtcaggcgtgggatgagacaaacgcggccataacagcggaatgacaccggtaaaccgaaaggcaggaacaggagagcgc

SEQ ID NO: 8 - DNA, 84 bp, Artificial Sequence
  Synthetic oligonucleotide use to construct a plasmid
cagttccgggtaggcagttcgctccaagctggactgtatgcacgaaccccccgttcagtccgaccgctgcgccttatccggtaa

SEQ ID NO: 9 - DNA, 85 bp, Artificial Sequence
  Synthetic oligonucleotide use to construct a plasmid
cgatagttaccggataaggcgcagcggtcggactgaacggggggttcgtgcatacagtccagcttggagcgaactgcctacccgg

SEQ ID NO: 10 - DNA, 87 bp, Artificial Sequence
  Synthetic oligonucleotide use to construct a plasmid
ctatcgtcttgagtccaacccggaaagacatgcaaaagcaccactggcagcagccacaggtaattgatttagaggagttagtcttga

SEQ ID NO: 11 - DNA, 79 bp, Artificial Sequence
  Synthetic oligonucleotide use to construct a plasmid
aagactaactcctctaaatcaattacctgtggctgctgccagtggtgcttttgcatgtctttccgggttggactcaaga

SEQ ID NO: 12 - DNA, 77 bp, Artificial Sequence
  Synthetic oligonucleotide use to construct a plasmid
agtcatgcgccggttaaggctaaactgaaaggacaagttttggtgactgcgctcctccaagccagttacctcggttc

SEQ ID NO: 13 - DNA, 74 bp, Artificial Sequence
  Synthetic oligonucleotide use to construct a plasmid
gaggtaactggcttggaggagcgcagtcaccaaaacttgtcctttcagtttagccttaaccggcgcatgacttc

SEQ ID NO: 14 - DNA, 76 bp, Artificial Sequence
  Synthetic oligonucleotide use to construct a plasmid
aaagagttggtagctcagagaaccttcgaaacaccgccctgcaaggcggttttttcgttctcagggcaagagatta

SEQ ID NO: 15 - DNA, 87 bp, Artificial Sequence
  Synthetic oligonucleotide use to construct a plasmid
cgcgcgtaatctcttgccctgagaacgaaaaaaccgccttgcagggcggtgtttcgaaggttctctgagctaccaactctttgaacc

SEQ ID NO: 16 - DNA, 37 bp, Artificial Sequence
  Synthetic oligonucleotide use to construct a plasmid
cgcgcggaccaaaacgatctcaagaagatcatcttat

SEQ ID NO: 17 - DNA, 33 bp, Artificial Sequence
  Synthetic oligonucleotide use to construct a plasmid
taataagatgatcttcttgagatcgttttggtc

SEQ ID NO: 18 - DNA, 27 bp, Artificial Sequence
  PCR primer
cggggatcctttatttcgattcactcc

SEQ ID NO: 19 - DNA, 41 bp, Artificial Sequence
  PCR primer
gggaattccatatgaatgccctccttataatcaaatgttac

(19) United States
(12) Patent Application Publication
(10) Pub. No.: US 2004/0259252 A1
(43) Pub. Date: Dec. 23, 2004

(54) Vector containing an enhanced p15a origin of replication

(76) Inventors: Mitchell C. Sanders, West Boylston, MA (US); Maureen A. Hamilton, Littleton, MA (US)

Correspondence Address: HAMILTON, BROOK, SMITH & REYNOLDS, P.C., 530 VIRGINIA ROAD, P.O. BOX 9133, CONCORD, MA, 01742-9133, US

(21) Appl. No.: 10/484,232

(22) PCT Filed: Jul. 18, 2002
(86) PCT No.: PCT/US02/23089

Related U.S. Application Data
(60) Provisional application No. 60/306,344, filed on Jul. 18, 2001.

(51) Int. Cl. C12N 15/85; C12N 15/74

(52) U.S. Cl. 435/455; 435/471; 435/320.1

(57) ABSTRACT

A molecular expression and cloning system which employs an oligonucleotide which converts a low copy number plasmid into a more efficient high copy number plasmid is disclosed. A high copy number plasmid can be used to facilitate more efficient expression or co-expression of proteins.

### RELATED APPLICATIONS

This application claims the benefit of U.S. Provisional Application No. 60/306,344, titled “A Vector Containing an Enhanced p15A Origin of Replication” filed on Jul. 18, 2001, the entire teachings of which are incorporated herein by reference.

### BACKGROUND OF THE INVENTION

Commonly in biotechnology, a gene or its product is of interest and needs to be expressed by living cells for study or use. There are many different expression systems available to the practitioner, including systems that rely upon prokaryotic and eukaryotic cells. Within each of these broad categories there are many specific expression systems designed for particular uses. However, prokaryotic systems (such as E. coli cultures) are often employed due to their ease and simplicity of use.

Many expression systems employ plasmids, which are small, autonomously replicating DNA molecules commonly isolated from prokaryotic cells. Naturally occurring plasmids can be engineered to serve as vectors to introduce exogenous genes into host cells. An efficient expression vector often combines a strong and tightly regulated promoter from a prokaryotic plasmid and at least one exogenous gene that provides the instructions for the cell to synthesize a desired protein.

A critical parameter in an expression system is the plasmid copy number that can be supported within a host cell. The copy number refers to the number of plasmids present in a host cell as a result of plasmid replication after transformation of the cell by the engineered plasmid. Production of proteins and other uses of the expression system are often benefitted by choosing a vector that has a high copy number.

A portion of a plasmid known as the origin of replication is critical in determining the copy number of a plasmid vector. The origin of replication contains sequences which regulate the replication of the plasmid as an autonomous unit.

Plasmids containing the p15A origin of replication are often used because they can be readily maintained in E. coli cells. Unfortunately, plasmids containing this p15A origin of replication generally have a low copy number. This low copy number presents difficulties when attempting to perform certain biological techniques, such as purifying DNA or cloning a particular nucleotide sequence or expressing a desired protein.

Therefore, a need exists for a vector having a high copy number, which can be used in expression systems like E. coli systems.

### SUMMARY OF THE INVENTION

The present invention pertains to a molecular cloning and expression system comprising a plasmid having a high copy number. This invention is based in part on the finding that key mutations in a p15A origin of replication (hereinafter “ori”) nucleotide sequence operatively linked within a plasmid results in the plasmid being converted from a low copy number to a high copy number plasmid. This type of high copy number plasmid can be used for more efficient cloning, DNA and RNA purification, protein expression, and co-expression heretofore unrealized.

In one embodiment of the present invention, an enhanced p15A ori is disclosed. In a particular aspect of the current invention, a low copy number pACYC184 plasmid, comprising a p15A ori, is converted into a high copy number plasmid. This conversion is due to key mutations introduced into the p15A ori nucleotide sequence. The enhanced p15A ori can be isolated and inserted into other low copy number plasmids, thereby converting them into high copy number plasmids.

In another embodiment, a method for constructing a plasmid having an enhanced p15A ori is disclosed. In one aspect of the invention, a plasmid is disclosed which comprises, among other nucleotide sequences, an enhanced p15A ori, a strong promoter, a terminator, and lacI^{q} gene. This plasmid can be used for the expression and/or co-expression of one or more proteins. Also this plasmid is beneficial in isolating nucleic acids of interest.

In yet another embodiment, the present invention pertains to a method for using an enhanced p15A ori in the expression and/or co-expression of proteins. A plasmid comprising the enhanced p15A ori and at least one nucleotide sequence encoding an exogenous protein (e.g., homologous and/or heterologous protein) is used to express the encoded polypeptide. More than one protein can be encoded within the plasmid, thus allowing for co-expression. In this embodiment, a host cell is transformed by a plasmid of this invention, which comprises the enhanced p15A ori sequence and one or more polypeptide encoding nucleic acid sequences. Under suitable conditions, the host cell expresses the homologous/heterologous protein in a manner that is enhanced or more efficient (e.g., a higher copy number of plasmids) than if the plasmid contained a native (non-mutated) p15A ori.

In still another embodiment, the present invention pertains to a method for using an enhanced p15A ori in the purification of a cloned nucleic acid, or fragment thereof. A plasmid comprising the enhanced p15A ori and at least one other nucleotide sequence is used to produce copies of that nucleic acid. In this embodiment, a host cell is transformed by this plasmid comprising an enhanced p15A ori sequence and a cloned nucleotide sequence. Under suitable conditions, the host cell makes copies of the plasmid and cloned nucleic acid in a manner that is more efficient than if the plasmid had contained a non-enhanced p15A ori.

### BRIEF DESCRIPTION OF THE DRAWINGS

The invention will be more fully understood from the following detailed description taken in conjunction with the accompanying drawings, in which:

FIG. 1 is a depiction of a genetic map of the pACYC184 plasmid.

FIG. 2A-B is a DNA sequence of the pACYC184 plasmid (SEQ ID NO. 1).

FIG. 3 is a DNA sequence of the enhanced mutated p15A ori (SEQ ID NO. 4), wherein the base substitutions appear in bold.

FIG. 4A-D is a DNA sequence alignment of the original p15A ori and the enhanced mutated p15A ori, wherein the top nucleotide sequence is the original p15A ori (SEQ ID NO. 5), wherein the base substitutions appear in bold.

FIG. 5 is a photograph of an agarose gel comparing the non-mutated p15A ori and the enhanced p15A ori. This 0.7% agarose gel was electrophoresed at 80 volts for 60 minutes. The gel was then stained for 10 minutes in 2 ug/ml ethidium bromide and de-stained for 25 minutes in water.

FIG. 6 is a depiction of a genetic map of the pECI-073 plasmid.

FIG. 7 is a photograph of an agarose gel analysis for copy number determination for pECI-073.

FIG. 8 is a depiction of a genetic map of the pECI-074 plasmid.

FIG. 9 is a photograph of an agarose gel analysis of the enhanced pECI-074 plasmid.

FIG. 10 is a depiction of a genetic map of the pECI-075 plasmid.

FIG. 11 is a schematic representation for the construction of plasmids described herein.

FIG. 12 is a depiction of a genetic map of the pECI-084 plasmid.

FIG. 13 is a depiction of a genetic map of the pECI-122 plasmid.

### DETAILED DESCRIPTION OF THE INVENTION

The present invention pertains to a molecular expression and cloning system which employs an oligonucleotide used to convert a low copy number plasmid into a more efficient high copy number plasmid. The high copy number plasmid can be used to facilitate more efficient expression or co-expression of proteins. The ease of nucleic acid cloning and purification is also enhanced with a high copy number plasmid.

The molecular cloning and expression system of the present invention comprises a high copy number plasmid whose genome includes a mutated p15A origin of replication sequence (ori) that is operatively-linked therein. Key mutations in this p15A ori convey the property of high copy number to an otherwise low copy number plasmid. This mutated p15A ori is referred to herein as “enhanced p15A ori.”

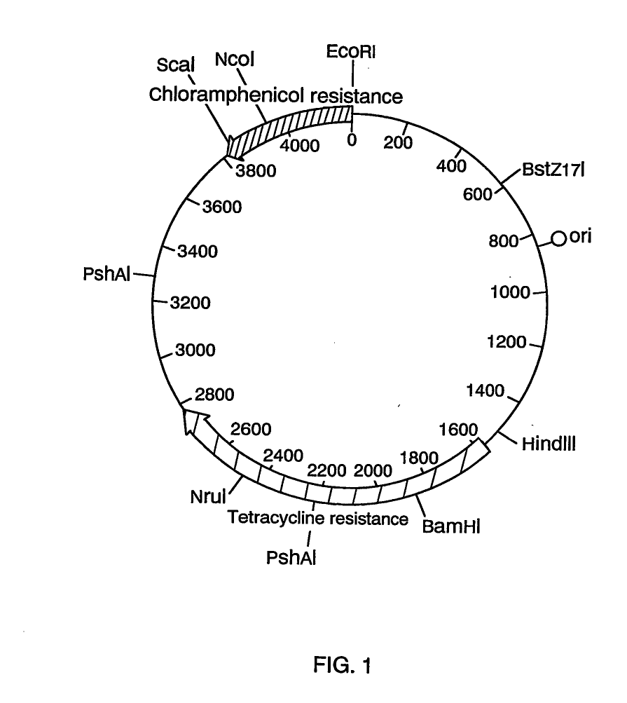

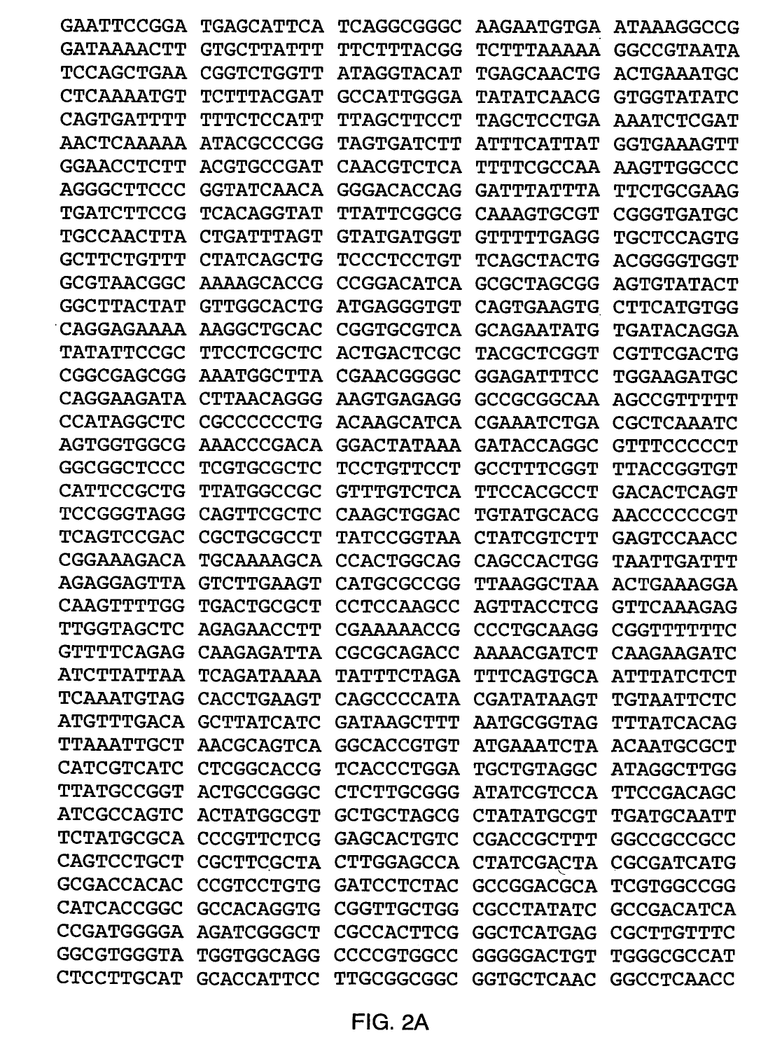

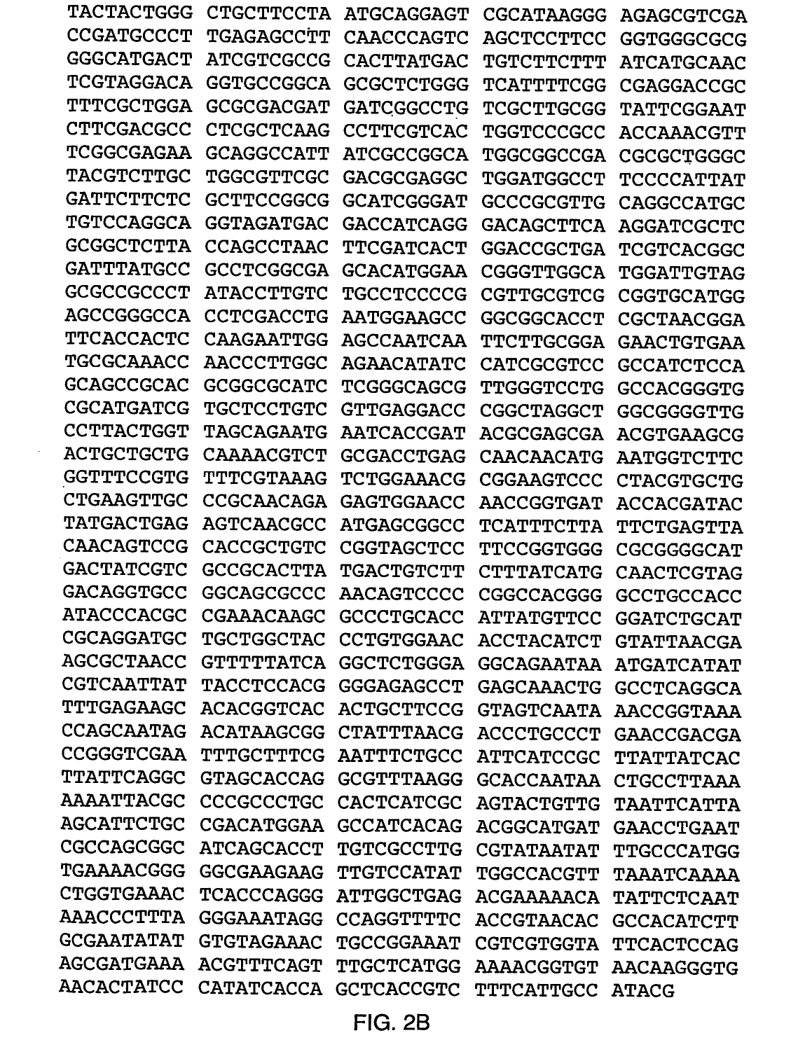

In general, this invention is due in part to the discovery that certain key mutations in the native p15A ori lead to enhanced cloning and expression by the vector system used. Mutations were introduced into a native p15A ori locus housed within a low copy number plasmid, specifically, the pACYC184 plasmid (catalog no. 401M, lot no. 6B, New England Biolabs, Beverly, Mass.). See FIG. 1 for the genetic map of the pACYC184 plasmid. This plasmid typically comprises a low copy number p15A ori locus. The locus of the p15A ori sequence comprises approximately a 1.5 Kb oligonucleotide which is positioned within an EcORI-HindIII restriction site (corresponding to sequence position about 1 to about 1523 of the nucleic acid sequence of pACYC184 (SEQ ID NO. 1 shown in FIG. 2)).

Random mutations can be introduced into an oligonucleotide during a Polymerase Chain Reaction (hereinafter “PCR”) using a Taq DNA polymerase that has reduced fidelity. Reaction conditions can promote an environment wherein the polymerase suffers this reduced fidelity. Mutations were introduced into the native p15A ori locus of pACYC184 during a PCR reaction cycle employing reduced Taq polymerase fidelity conditions. Such conditions can include high and pool-biased dNTP concentrations, for example, 1 mM each of dTTP, dCTP, dGTP, and 200 μM of dATP. Additionally, a relatively high concentration of MgCl2, for example about 6.1 mM, was used in the presence of about 0.5 mM MnCl₂. Approximately 25 PCR cycles were conducted using an initial target plasmid amount of about 1 ng. An increased number of PCR cycles and/or an alternative DNA polymerase with reduced 3′-5′ exonuclease activity can further reduce fidelity. One skilled in the art will appreciate that there are other reaction conditions which promote low fidelity in the polymerase. These equivalent conditions are considered to be within the scope of the present invention.

Using the low copy number pACYC184 plasmid, random mutations were introduced employing PCR primers that were designed having a forward primer comprising an EcORI site and a reverse pruner containing a Hinarm site. The forward primer sequence used was: CCGGAGAATTCCGGATGAGCATTCATCAGG; SEQ ID NO. 2. The reverse primer sequence was: CCCGAAAGCTTATCGATGATAAGCTGTCAAACATG, SEQ ID NO. 3. Both primers were purchased from Life Technologies in Rockville, Md. (catalog no. 10336-022, forward primer lot no. R7654A02, reverse primer lot no. R7654A01).

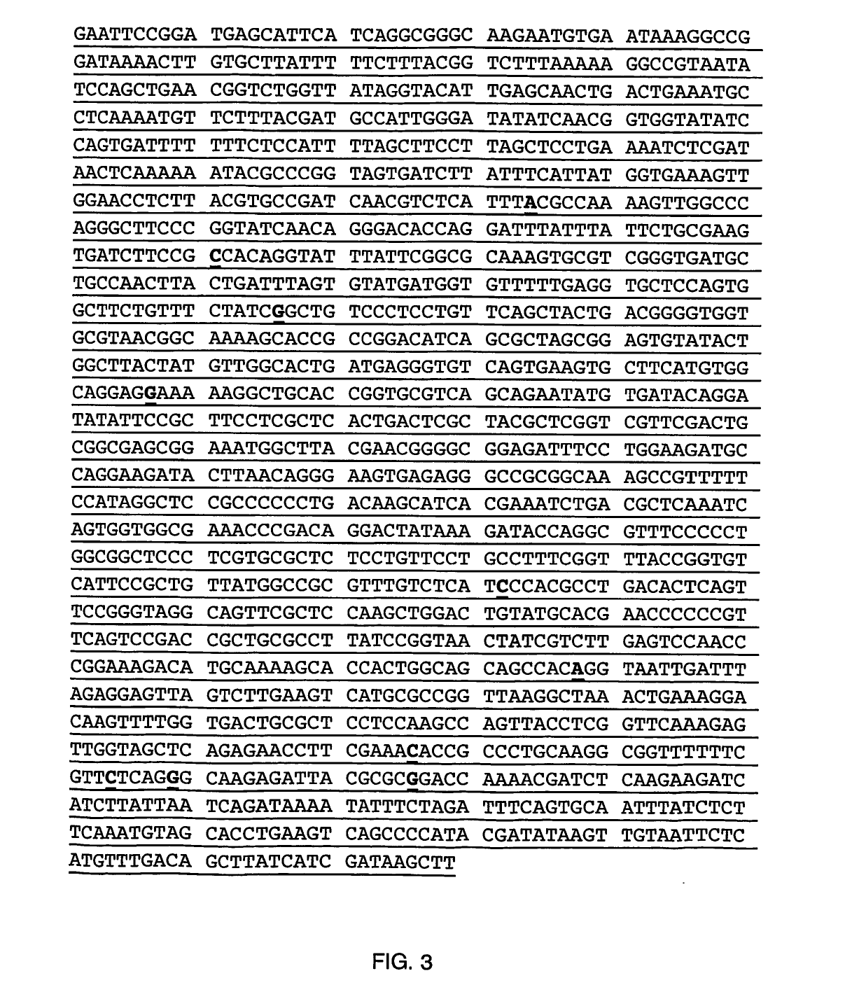

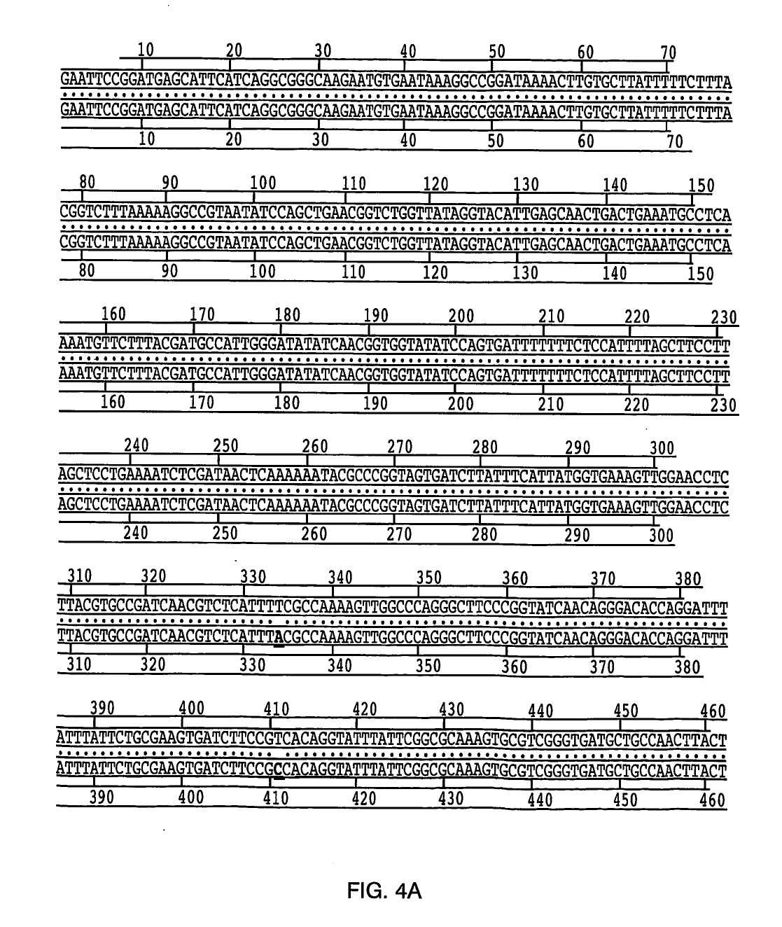

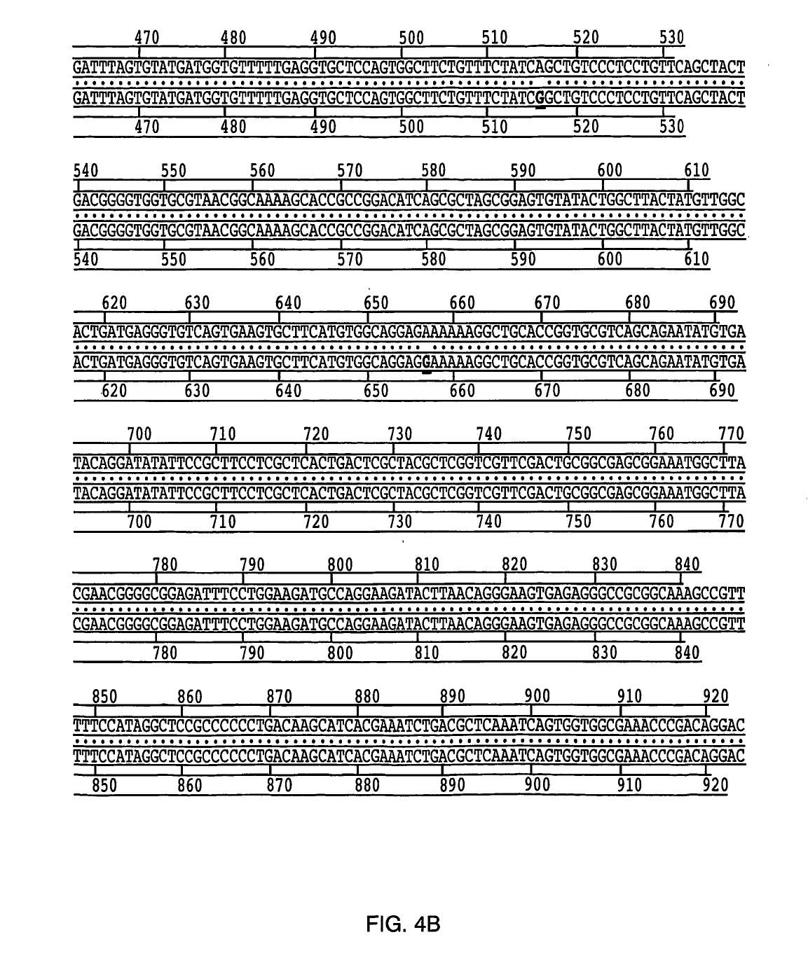

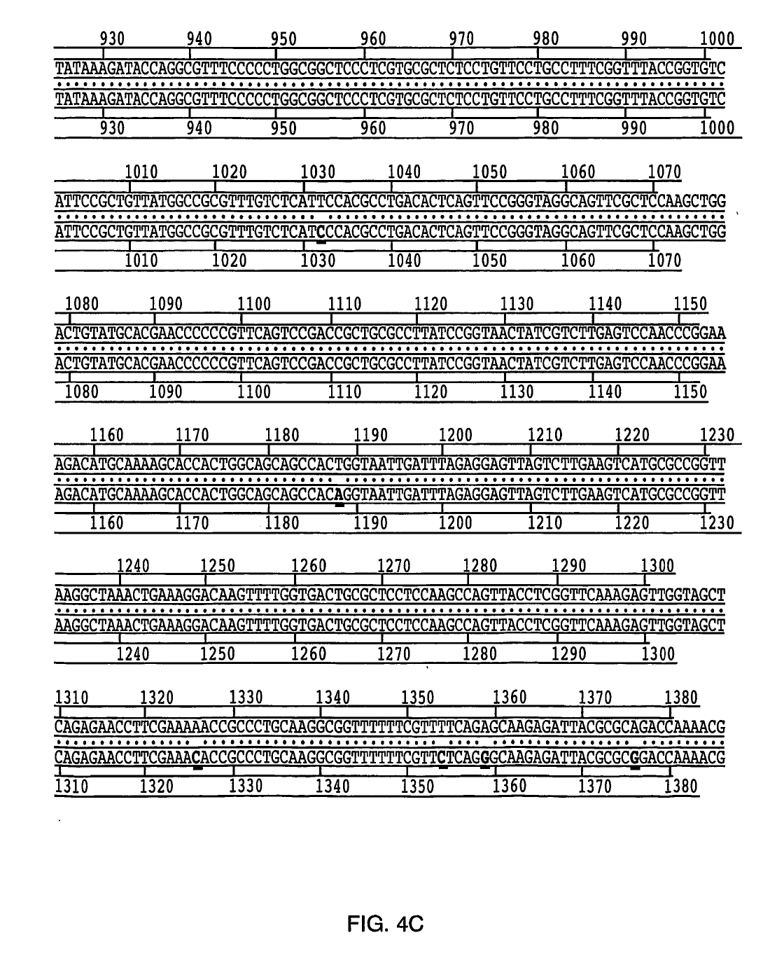

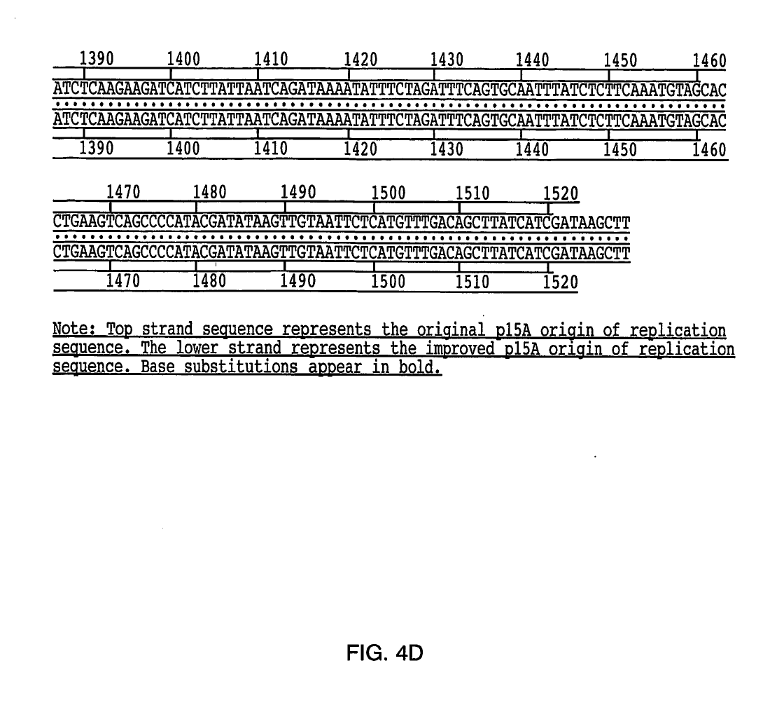

These primers used in the PCR amplification of the native p15A ori locus of pACYC184 were subject to conditions suitable for promoting mutational events. The PCR procedure yielded a 1.5 Kb DNA product. See FIGS. 3 (SEQ ID NO. 4) and 4 (SEQ ID NO. 5). This 1.5 Kb DNA product comprised a mutated p15A on sequence. Following PCR, this mutated DNA product was purified using Qiagen's QIA quick PCR Purification Kit (catalog no. 28104, lot no. HQO31/Q02/222, Valencia, Calif.). After this purification step, a double digest of the purified PCR DNA product was accomplished using EcORI and HindIII, which removed any overhanging ends of the forward and reverse primers. The digested, mutated p15A ori DNA was next subjected to agarose gel electrophoresis followed by gel purification using BIO 101's Geneclean Spin Kit (catalog no. 1101-600, lot no. 1101-600-623-2, Carlsbad, Calif.) yielding a highly purified 1.5 Kb DNA containing the mutated p15A ori.

A second pACYC184 plasmid was digested using EcORI and HindIII producing a 2.7 Kb linearized plasmid. (The EcORI/HindIII excised fragment comprised the native p15A ori nucleotide sequence.) The 2.7 Kb plasmid product was next subjected to agarose gel electrophoresis followed by gel purification using BIO 101's Geneclean Spin Kit yielding a highly purified 2.7 Kb plasmid.

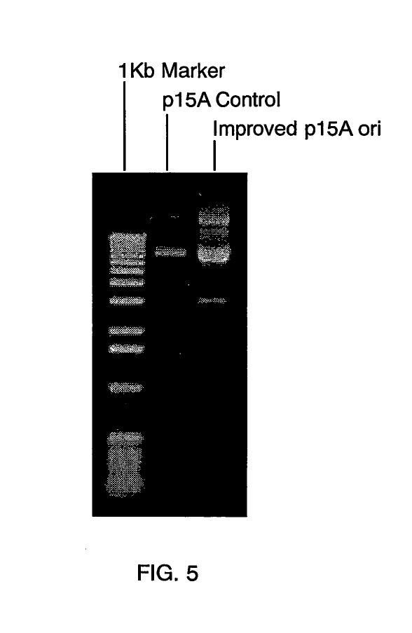

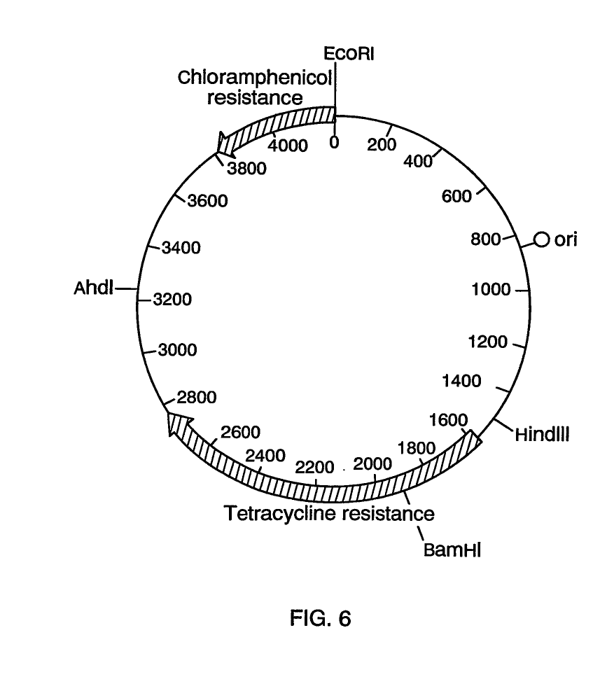

At this point in the method for synthesizing a nascent high copy number plasmid, a purified 1.5 Kb mutated p15A ori DNA sequence and a purified 2.7 Kb pACYC184 fragment have been formed. The purified 1.5 Kb DNA comprising the mutated p15A ori product was ligated to the purified 2.7 Kb pACYC184 fragment forming a library of newly constructed plasmids. Ligations were performed using 3 Weiss units of T4 DNA ligase (New England Biolabs cat #M0202S) in a volume of 10 μl at 12° C. for 16 hours. This newly created plasmid library was then used to transform electrocompetent E. coli BL21(DE3) cells (Novagen cat # 20 69387-3) using methods well known in the art. The transformed cells were subsequently plated out onto culture plates using techniques well known to practitioners in the art. The transformed colonies were next screened for an operationally enhanced p15A ori. Approximately 10 ml of 2×LB CM50 bacterial growth media, consisting of tryptone 20 g/L (Difco cat # DF0123-08), yeast extract 10 g/L (Difco cat # DF0127-08) sodium chloride 10 g/L (J. T. Baker cat #M216-25 18) and chloramphenicol 50 μg/ml (Sigma cat # C0378), was inoculated for each transformant colony. Each culture was permitted to grow overnight with agitation at approximately 37° C. On the following day, about 8 ml of each culture was processed in order to obtain plasmid DNA by methods well known in the art using Qiagens QIAprep Spin Miniprep Kit (cat no. 27106, lot no. IGQ025/Q01/421). A standard volume of each miniprep (about 15-20 ,μl), which contained plasmid DNA, was analyzed using a 0.7% agarose gel electrophoresis and compared to a normal (non-mutated) pACYC184 miniprep (same volume loaded). Ethidium bromide staining of the gel was used to determine which, if any, transformant colony contained an enhanced mutation in the p15A ori locus by observing differences in band intensity which are attributable to changes in copy number. FIG. 5 shows such a gel. The first lane contains molecular weight markers used to estimate the size of the samples. The second lane contains a controlled p15A sample that was not subjected to mutagenic conditions. The last lane shows the results of a plasmid (hereinafter referred to as “pECI-073”) with an enhanced (or improved) p15A ori. See FIG. 6, which depicts the genetic map for the pECI-073 plasmid.

The pECI-073 plasmid was sequenced to determine the nucleotide position of the mutations. Table 1 illustrates the findings of this sequence analysis.

TABLE 1
Base substitutions of plasmid pECI-073
as determined by DNA sequencing.

| Mutation Number | Base Number | Substitution |
|---|---|---|
| 1 | 334 | T to an A |
| 2 | 441 | T to a C |
| 3 | 516 | A to a G |
| 4 | 657 | A to a G |
| 5 | 1032 | T to a C |
| 6 | 1188 | T to an A |
| 7 | 1326 | A to a C |
| 8 | 1354 | T to a C |
| 9 | 1359 | A to a G |
| 10 | 1376 | A to a G |

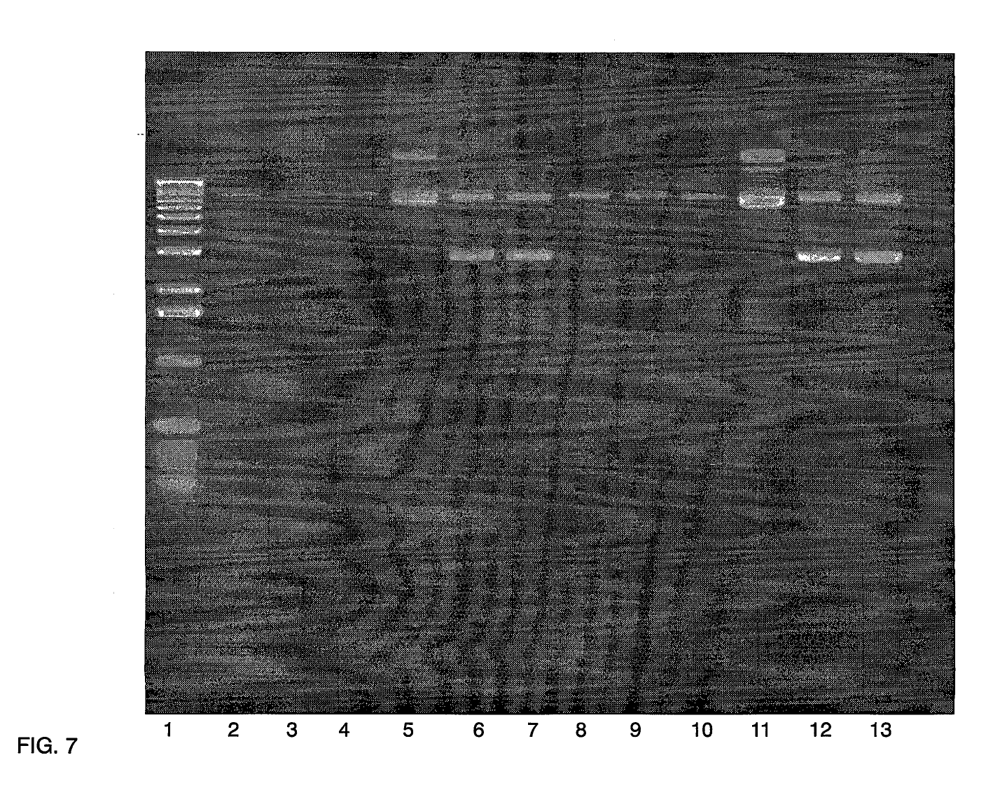

To verify the increase copy number of pECI-073 the following procedure was performed. Overnight cultures of E. coli strain BL21(DE3) containing either pACYC184 or plasmid pECI073 were grown in 10 ml 2×LB (CMS0) in triplicate. On the following morning, the optical density (at 600 nanometers) of each of the six cultures was measured, and the cultures were all normalized to a value of 0.96 using sterile 2×LB. For each of the normalized cultures, 1.5 ml was extracted, and the plasmid DNA was purified using the Qiagen QIAprep Spin Miniprep Kit. The final product for each preparation was eluted in 30 μl TE pH 7.5 (TE: 10 mM Tris-HCl, 1 mM EDTA). Two samples of each plasmid preparation (one of 3 μl and one of 6 μl) were loaded onto a 0.7% agarose gel which was subjected to electrophoresis at 80 V for one hour, then stained with ethidium bromide. An image of the stained agarose gel is depicted in FIG. 7 and was subsequently digitized. Referring to FIG. 7, lane 1 represents a 1 Kb ladder, lanes 2-4 represent 3 μl of control (i.e., pACYC184), lanes 5-7 represent 3 μl of pECI-073 plasmid comprising the enhanced ori, lanes 8-10 represent 6 μl of control, and lanes 11-13 represent 6 μl of pECI-073 plasmid with the enhanced ori. The relative staining intensities of the plasmid bands was determined using the NIH Image program. Table 2 illustrates the quantitation of the increase in copy number.

TABLE 2

| | | Band density after | | |
|---|---|---|---|---|
| | Volume | Background | Mean band | Aggregate |
| Sample ID | Loaded | subtraction | density | Mean |
| PACYC184#1 | 3 μl | 39705.6 | 30958 | 31661.3 |
| PACYC184#2 | 3 μl | 25126.2 | | |
| PACYC184#3 | 3 μl | 284042.1 | | |
| PACYC184#1 | 6 μl | 36232.4 | 32364.6 | |
| PACYC184#2 | 6 μl | 26491.1 | | |
| PACYC184#3 | 6 μl | 34370.2 | | |
| pECI-073 #1 | 3 μl | 155410.2 | 122239.5 | 14123.6 |
| pECI-073 #2 | 3 μl | 105406.0 | | |
| pECI-073 #3 | 3 μl | 105406.0 | | |
| pECI-073 #1 | 6 μl | 211680.5 | 160807.7 | |
| pECI-073 #2 | 6 μl | 134068.4 | | |
| pECI-073 #3 | 6 μl | 136674.1 | | |

Based upon the aggregate mean band densities shown in Table 2, it was calculated that the copy number of pECI-073 is about 4.47 fold higher than that of pACYC184. Based upon a published copy number of 18-22 for plasmid pACYC184, it is estimated that the improved vector pECI-073 has a copy number of about 80-98 per cell. See, Chang and Cohen, Journal of Bacteriology, 134:1141-1156 (1978).

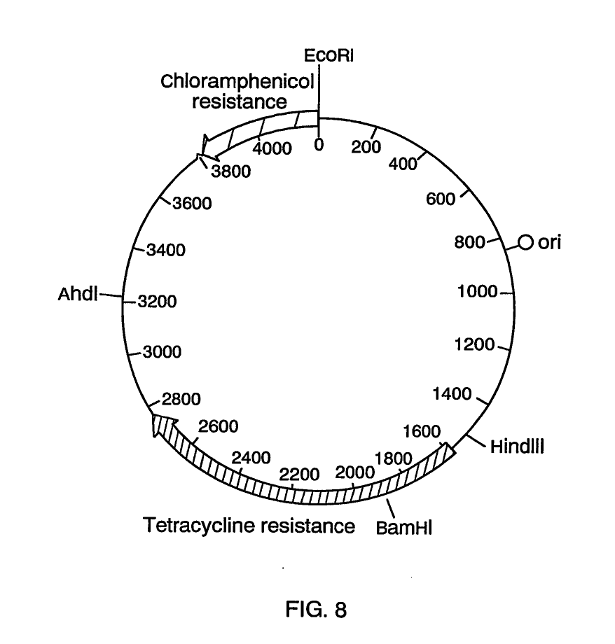

In one embodiment of the present invention, a plasmid is constructed which comprises an enhanced p15A ori using plasmid pECI-073. The oligonucleotide comprising the enhanced p15A ori is excised from pECI-073. The pACYC184 plasmid is prepared to receive the enhanced p15A ori by removing a segment of oligonucleotide that comprises a native p15A ori. The enhanced p15A ori from pECI-073 is ligated to the pACYC184 plasmid yielding a nascent pECI-074 plasmid. See FIG. 8.

To illustrate this embodiment, the pECI-073 plasmid was first digested using HindIII and SacII which yielded a 0.7 Kb fragment. This 0.7 Kb DNA fragment comprised p15A ori mutations 5 through 10, as depicted in Table 1. The fragment was subsequently subjected to agarose gel electrophoresis and gel purified using a BIO 101 Geneclean Spin Kit.

Next, plasmid pACYC184 was digested using HindIII and SacII yielding a 3.5 Kb DNA fragment. This fragment was subsequently subjected to agarose gel electrophoresis and purified by gel purification using a BIO 101 Geneclean Spin Kit.

The 0.7 Kb gel purified DNA fragment (from pECI-073) was then ligated to the 3.5 Kb pACYC184 fragment to form the new plasmid, pECI-074. The ligated product was then transformed into BL21(DE3) cells and plated for transformants on chloramphenicol-containing plates. The nascent plasmid pECI-074 has a nucleotide sequence that encodes for chloramphenicol resistance (from the pACYC184 fragment). Several transformants were obtained from the transformation.

The transformed colonies were then screened for an operationally enhanced p15A ori gene. Approximately 10 ml of 2×LB CM50 culture was prepared for each transformant colony. The culture was permitted to grow overnight with agitation at approximately 37° C. On the following day, the optical density (at 600 nm) of each culture was measured, and the cultures, including the control, were all normalized to a value of about 1.03 using 2×LB. About 1.5 ml of each culture was processed in order to obtain plasmid DNA by methods well known in the art using, for example, Qiagens QIAprep Spin Miniprep Kit (catalog no. 27106, lot no. IGQ025/Q01/421). A standard volume of each miniprep (about 8.5 pl) was analyzed by 0.7% agarose gel electrophoresis and compared to a normal (non-mutated) pACYC184 miniprep (same volume loaded). See FIG. 9. Ethidium bromide staining of the gel was used to determine which, if any, transformant colonies contain a copy number enhancing mutation in the p15A ori locus by observing differences in band intensity.

Bands having high intensity, when compared to a control, indicated transformants having a enhanced p15A ori. Such a comparison of the band intensity for the transformants (comprising pECI-074) with that of the pACYC184 control clearly indicated that the transformants have an enhanced copy number due to the improved p15A ori. This data indicates that only those p15A ori mutations numbered 5-10 in Table 1 are required to create an improved vector having an enhanced p15A ori, e.g, pECI-074.

In one embodiment, a method for making a vector comprising an enhanced synthetic p15A ori locus is disclosed. The strategy for creating this new vector comprises replacing the original p15A ori in pACYC184 with an enhanced synthetic p15A ori. This can be accomplished by designing a p15A ori using complementary pairs of oligonucleotides that contain appropriate base substitutions. These complementary oligonucleotides are annealed together, and the annealed oligonucleotide complex is ligated into a vector. The synthetic, single stranded oligonucleotides are preferably designed to anneal in pairs, and concatenate to form a synthetic double stranded DNA. The newly created double stranded DNA may have overhanging ends which are compatible with restriction enzymes. In one particular embodiment, the nascent DNA contains restriction sites for AseI and BssSI. The restriction enzymes will remove these overhanging ends provided there is a restriction site contained within the DNA substrate. A plasmid such as pACYC184 is next digested with restriction enzymes, for example, AseI and BssSI, in order to remove a region of the plasmid, e.g., the native ori nucleotide sequence. The region can now be occupied by the complementary oligonucleotides described below.

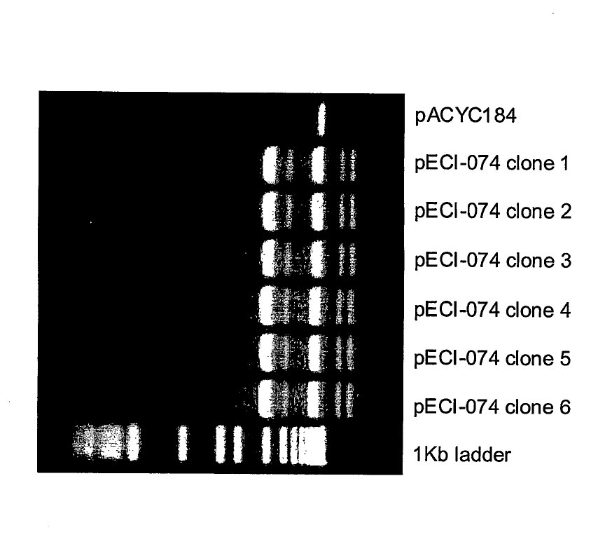

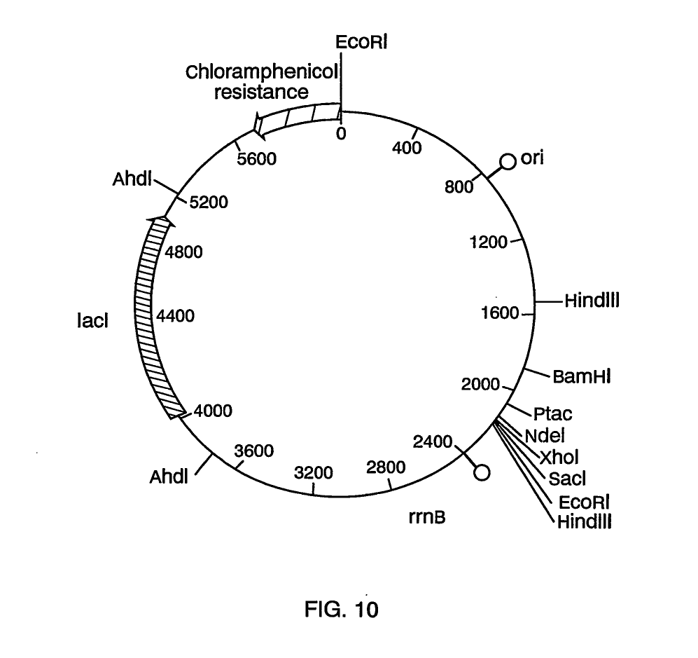

To exemplify the construction of such a plasmid using the above described method, synthetic oligonucleotides were inserted into a linearized pACYC184 in order to produce a nascent plasmid, pECI-075. See FIG. 10. The oligonucleotides were first annealed and then ligated under conditions well known in the art using T4 DNA ligase (New England Biolabs). The oligonucleotides used were those listed in Table 3 and can be ordered from Life Technologies. These oligonucleotides were annealed in pairs (SEQ ID #6 with #7 to form pair A, #8 with #9 to form pair B, #10 with #11 to form pair C, #12 with #13 to form pair D, #14 with #15 to form pair E, and #16 with #17 to form pair F). These annealed pairs A through F have complementary overhanging ends that allow them to anneal with the other pairs to form a linear molecule, in which the pairs are ordered A-F. The paired and annealed oligos were then ligated together to form a 450 base-pair insert containing the six mutations (see above) known to enhance the p15A ori. A strong promoter such as the lacZ or T7 promoters, a multiple cloning site or “MCS” such as that found in the commonly used pUC vectors, and a transcriptional terminator such as rrnB were added to this nascent plasmid. The selection marker, or markers, can be any antibiotic resistance marker, for example, chloramphenicol acetyl transferase that is present in plasmid pACYC184. A lacI^{q} gene can be cloned into a PshAI restriction site of the plasmid allowing for induction by the addition of about 1-5 mM isopropyl β-D-thiogalacto-pyranoside (IPTG de-represses the lac repressor protein).

TABLE 3
Synthetic oligonucleotides used to construct plasmid pECI-075

| SEQ | | |
|---|---|---|
| ID | Oligonucleotide sequence | Comments |
| #6 | TCGTGCGCTCTCCTGTTCCTGCCTTTCGGTTTACCGGTGTC | Complementary with |
| | ATTCCGCTGTTATGGCCGCGTTTGTCTCATCCCACGCCTGA | #7, contains mutation 5 |
| | CACT | from Table I |
| #7 | AACTGAGTGTCAGGCGTGGGATGAGACAAACGCGGCCAT | Complementary with |
| | AACAGCGGAATGACACCGGTAAACCGAAAGGCAGGAACA | #6, contains mutation 5 |
| | GGAGAGCGC | |
| #8 | CAGTTCCGGGTAGGCAGTTCGCTCCAAGCTGGACTGTATG | Complementary with |
| | CACGAACCCCCCGTTCAGTCCGACCGCTGCGCCTTATCCG | #9 |
| | GTAA | |
| #9 | CGATAGTTACCGGATAAGGCGCAGCGGTCGGACTGAACG | Complementary with |
| | GGGGGTTCGTGCATACAGTCCAGCTTGGAGCGAACTGCCT | #8 |
| | ACCCGG | |
| #10 | CTATCGTCTTGAGTCCAACCCGGAAAGACATGCAAAAGCA | Complementary with |
| | CCACTGGCAGCAGCCACAGGTAATTGATTTAGAGGAGTTA | #11, contains mutation |
| | GTCTTGA | 6 from Table 1 |
| #11 | AAGACTAACTCCTCTAAATCAATTACCTGTGGCTGCTGCC | Complementary with |
| | AGTGGTGCTTTTGCATGTCTTTCCGGGTTGGACTCAAGA | #10, contains mutation |
| | | 6 |
| #12 | AGTCATGCGCCGGTTAAGGCTAAACTGAAAGGACAAGTTT | Complementary with |
| | TGGTGACTGCGCTCCTCCAAGCCAGTTACCTCGGTTC | #13 |
| #13 | GAGGTAACTGGCTTGGAGGAGCGCAGTCACCAAAACTTGT | Complementary with |
| | CCTTTCAGTTTAGCCTTAACCGGCGCATGACTTC | #12 |
| #14 | AAAGAGTTGGTAGCTCAGAGAACCTTCGAAACACCGCCCT | Complementary with |
| | GCAAGGCGGTTTTTTCGTTCTCAGGGCAAGAGATTA | #15, contains |
| | | mutations 7, 8, & 9 |
| | | from Table 1 |
| #15 | CGCGCGTAATCTCTTGCCCTGAGAACGAAAAAACCGCCTT | Complementary with |
| | GCAGGGCGGTGTTTCGAAGGTTCTCTGAGCTACCAACTCT | #14, contains |
| | TTGAACC | mutations 7, 8 & 9 |
| #16 | CGCGCGGACCAAAACGATCTCAAGAAGATCATCTTAT | Complementary with |
| | | #17, contains mutation |
| | | 10 |
| #17 | TAATAAGATGATCTTCTTGAGATCGTTTTGGTC | Complementary with |
| | | #16, contains mutation |
| | | 10 |

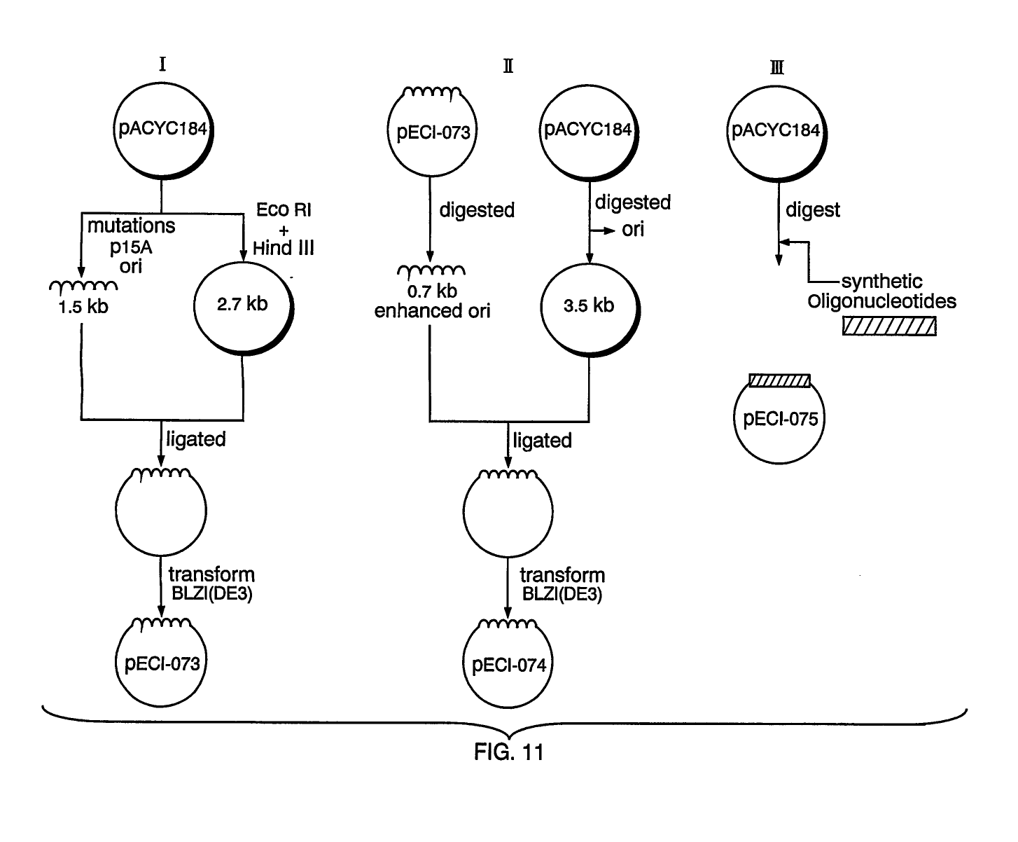

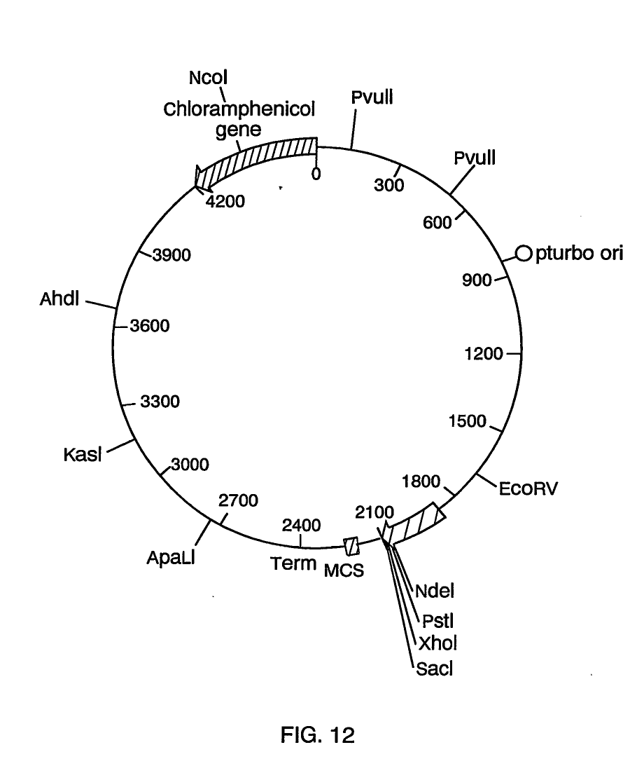

FIG. 11 is a schematic which shows the basic steps of producing the plasmids of the present invention.

To facilitate the understanding of the present invention, a number of terms and phrases are defined below:

As used herein, the terms “polynucleotide” and “oligonucleotide” are used interchangeably, and include polymeric forms of nucleotides of any length, either deoxyribonucleotides or ribonucleotides, or analogs thereof. Polynucleotides can have any three-dimensional structure, and can perform any function, known or unknown. The following are non-limiting examples of polynucleotides: a gene or gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide can comprise modified nucleotides, such as methylated nucleotides and nucleotide analogs. If present, modifications to the nucleotide structure can be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, any embodiment of this invention that is a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T), and uracil (U) for thymine when the polynucleotide is RNA. This, the term “polynucleotide sequence” is the alphabetical representation of a polynucleotide molecule. This alphabetical representation can be inputted into databases in a computer having a central processing unit and used for bioinformatics applications such as functional genomics and homology searching.

A “gene” includes a polynucleotide containing at least one open reading frame that is capable of encoding a particular polypeptide or protein after being transcribed and translated. Any of the polynucleotide sequences described herein may be used to identify larger fragments or full-length coding sequences of the gene with which they are associated. Methods of isolating larger fragment sequences are known to those of skill in the art, some of which are described herein.

A “gene product” includes an amino acid, e.g., peptide or polypeptide, generated when a gene is transcribed and then translated.

A “primer” includes a short polynucleotide, generally with a free 3′-OH group that binds to a target or “template” present in a sample of interest by hybridizing with the target, and thereafter promoting polymerization of a polynucleotide complementary to the target. A “polymerase chain reaction” (“PCR”) is a reaction in which replicate copies are made of a target polynucleotide using a “pair of primers” or “set of primers” consisting of “upstream” and a “downstream” primer, and a catalyst of polymerization, such as a DNA polymerase, typically a thermally-stable polymerase enzyme. Methods for PCR are well known in the art, and are taught, for example, in MacPherson et al., TRL Press at Oxford University Press (1991). All processes of producing replicate copies of a polynucleotide, such as PCR or gene cloning, are collectively referred to herein as “replication”. A primer can also be used as a probe in hybridization reactions, such as Southern or Northern blot analyses (see, for example, Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989).

The term “cDNAs” includes complementary DNA, that is mRNA molecules present in a cell or organism made into cDNA with an enzyme such as reverse transcriptase. A “cDNA library” includes a collection of mRNA molecules present in a cell or organism, converted into cDNA molecules with the enzyme reverse transcriptase, then inserted into “vectors” (other DNA molecules that can continue to replicate after addition of foreign DNA). Exemplary vectors for libraries include bacteriophage, viruses that infect bacteria, e.g., λ phage. The library can then be probed for the specific cDNA (and thus mRNA) of interest.

The term “polypeptide” includes a compound of two or more subunit amino acids, amino acid analogs, or peptidomimetics. The subunits may be linked by peptide bonds. In another embodiment, the subunit may be linked by other bonds, e.g. ester, ether, etc. As used herein the term “amino acid”, includes either natural and/or unnatural or synthetic amino acids, including glycine and both the D or L optical isomers, and amino acid analogs and peptidomunetics. A peptide of three or more amino acids is commonly referred to as an oligopeptide. Peptide chains of greater than three or more amino acids are referred to as a polypeptide or a protein.

“Hybridization” includes a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PCR reaction, or the enzymatic cleavage of a polynucleotide by a ribozyme.

Hybridization reactions can be performed under conditions of different “stringency.” The stringency of a hybridization reaction includes the difficulty with which any two nucleic acid molecules will hybridize to one another. Under stringent conditions, nucleic acid molecules at least about 60%, 65%, 70%, 75%, 80%, 85%, 90% or 95% identical to each other remain hybridized to each other, whereas molecules with low percent identity cannot remain hybridized. A preferred, non-limiting example of highly stringent hybridization conditions are hybridization in 6× sodium chloride/sodium citrate (SSC) at about 45° C., followed by one or more washes in 0.2×SSC, 0.1% SDS at 50° C., preferably at 55° C., more preferably at 60 C, and even more preferably at 65° C. An isolated nucleic acid molecule of the invention is at least 15, 20, 25, 30, 50, 100, 150, 200, 250, 300, 350, 400, 450, 500, 550, 600, 650, 700, 750, 800, 850, 900, 950, 1000, 1100, 1200, 1300, 1400, 1500, or more nucleotides in length and hybridizes under stringent conditions to a nucleic acid molecule corresponding to a nucleotide sequence of SEQ ID NO. 4.

When hybridization occurs in an antiparallel configuration between two single-stranded polynucleotides, the reaction is called “annealing” and those polynucleotides are described as “complementary”. A double-stranded polynucleotide can be “complementary” or “homologous” to another polynucleotide, if hybridization can occur between one of the strands of the first polynucleotide and the second. “Complementarity” or “homology” (the degree that one polynucleotide is complementary with another) is quantifiable in terms of the proportion of bases in opposing strands that are expected to hydrogen bond with each other, according to generally accepted base-pairing rules.

As used herein, the term “nucleic acid molecule” is intended to include DNA molecules, e.g., cDNA or genomic DNA, and RNA molecules, e.g., mRNA, and analogs of the DNA or RNA generated using nucleotide analogs. The nucleic acid molecule can be single-stranded or double-stranded, but preferably is double-stranded DNA.

The term “isolated nucleic acid molecule” includes nucleic acid molecules which are separated from other nucleic acid molecules which are present in the natural source of the nucleic acid. For example, with regards to genomic DNA, the term “isolated” includes nucleic acid molecules which are separated from the chromosome with which the genomic DNA is naturally associated. Preferably, an “isolated” nucleic acid is free of sequences which naturally flank the nucleic acid (i.e., sequences located at the 5′ and 3′ ends of the nucleic acid) in the genomic DNA of the organism from which the nucleic acid is derived. For example, in various embodiments, the isolated marker nucleic acid molecule of the invention, or nucleic acid molecule encoding a polypeptide marker of the invention, can contain less than about 5 kb, 4 kb, 3 kb, 2 kb, 1 kb, 0.5 kb or 0.1 kb of nucleotide sequences which naturally flank the nucleic acid molecule in genomic DNA of the cell from which the nucleic acid is derived. Moreover, an “isolated” nucleic acid molecule, such as a cDNA molecule, can be substantially free of other cellular material, or culture medium when produced by recombinant techniques, or substantially free of chemical precursors or other chemicals when chemically synthesized.

A nucleic acid molecule of the present invention, e.g. a nucleic acid molecule having the nucleotide sequence of SEQ ID NO. 4, or a portion or functional fragment thereof, can be isolated using standard molecular biology techniques and the sequence information provided herein. Using all or portion of the nucleic acid sequence of SEQ ID NO. 4 as a hybridization probe, a molecule comprising SEQ ID NO. 4 can be isolated using standard hybridization and cloning techniques as described in Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989.

Functional, or biologically active fragments of the nucleic acid molecules described herein are also encompassed by the present invention. Function, or activity can be determined by assays known to those of skill in the art. For example, in the present invention, fragments of the ori sequences can be produced and tested (as described herein) for their ability to increase plasmid copy number.

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to marker nucleotide sequences, or nucleotide sequences encoding a marker of the invention can be prepared by standard synthetic techniques, e.g, using an automated DNA synthesizer.

In another embodiment, an isolated nucleic acid molecule of the invention comprises a nucleic acid molecule which is a complement of the nucleotide sequence of SEQ ID NO. 4, or a portion thereof. A nucleic acid molecule that is complementary to such a nucleotide sequence is one which is sufficiently complementary to the nucleotide sequence such that it can hybridize to the nucleotide sequence, thereby forming a stable duplex.

The nucleic acid molecule of the invention, moreover, can comprise only a portion of the nucleic acid sequence of SEQ ID NO. 4 of the invention, or a fragment which can be used as a probe or primer. The probe/primer typically comprises substantially purified oligonucleotide. The oligonucleotide typically comprises a region of nucleotide sequence that hybridizes under stringent conditions to at least about 7 or 15, preferably about 20 or 25, more preferably about 50, 75, 100, 125, 150, 175, 200, 225, 250, 275, 300, 325, 350, 400 or more consecutive nucleotides of a marker nucleic acid, or a nucleic acid encoding a marker polypeptide of the invention.

Probes based on the nucleotide sequence of a nucleic acid molecule encoding enhanced p15A ori can be used to detect transcripts or genomic sequences corresponding to SEQ ID NO. 4. In other embodiments, the probe comprises a labeling group attached thereto, e.g., the labeling group can be a radioisotope, a fluorescent compound, an enzyme, or an enzyme cofactor. Such probes can be used as a part of a diagnostic test kit for identifying cells or tissue which misexpresses, e.g., over- or under-express, a polypeptide of the invention, or which have greater or fewer copies of a gene of the invention.

Another aspect of the invention pertains to vectors, preferably expression vectors, containing a nucleic acid encoding a marker protein of the invention (or a portion thereof. As used herein, the term “vector” includes a nucleic acid molecule capable of transporting another nucleic acid to which it has been linked. One type of vector is a “plasmid”, which includes a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced, e.g., bacterial vectors having a bacterial origin of replication and episomal mammalian vectors. Other vectors, e.g., non-episomal mammalian vectors, are integrated into the genome of a host cell upon introduction into the host cell, and thereby are replicated along with the host genome. Moreover, certain vectors are capable of directing the expression of genes to which they are operatively linked. Such vectors are referred to herein as “expression vectors.” In general, expression vectors of utility in recombinant DNA techniques are often in the form of plasmids. In the present specification, “plasmid” and “vector” can be used interchangeably as the plasmid is the most commonly used form of vector.

As used herein, “expression” includes the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgarno sequence and the start codon AUG (Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, which is operatively linked to the nucleic acid sequence to be expressed. Within a recombinant expression vector, “operatively linked” is intended to mean that the nucleotide sequence of interest is linked to the regulatory sequence(s) in a manner which allows for expression of the nucleotide sequence, e.g., in an in vitro transcription/translation system or in a host cell when the vector is introduced into the host cell. The term “regulatory sequence” is intended to include promoters, enhancers and other expression control elements, e.g., polyadenylation signals. Such regulatory sequences are described, for example, in Goeddel; Gene Expression Technology: Methods in Enzymology 185, Academic Press, San Diego, Calif. (1990). Regulatory sequences include those which direct constitutive expression of a nucleotide sequence in many types of host cells and those which direct expression of the nucleotide sequence only in certain host cells, e.g., tissue-specific regulatory sequences. It will be appreciated by those skilled in the art that the design of the expression vector can depend on such factors as the choice of the host cell to be transformed, the level of expression of protein desired, and the like. The expression vectors of the invention can be introduced into host cells to thereby produce proteins or peptides, including fusion proteins or peptides, encoded by nucleic acids as described herein, e.g., marker proteins, mutant forms of marker proteins, fusion proteins, and the like.

The recombinant expression vectors of the invention can be designed for expression of marker proteins in prokaryotic or eukaryotic cells. For example, proteins can be expressed in bacterial cells such as E. coli, insect cells (using baculovirus expression vectors) yeast cells or mammalian cells. Suitable host cells are discussed further in Goeddel, Gene Expression Technology: Methods in Enzymology 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in E. coli with vectors containing constitutive or inducible promoters directing the expression of either fusion or non fusion proteins. Fusion vectors add a number of amino acids to a protein encoded therein, usually to the amino terminus of the recombinant protein. Such fusion vectors typically serve three purposes: 1) to increase expression of recombinant protein, 2) to increase the solubility of the recombinant protein, and 3) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith, D. B. and Johnson, K S. (1988) Gene 67:31-40), pMAL (New England Biolabs, Beverly, Mass.) and pRIT5 (Pharmacia, Piscataway, N.J.) which fuse glutathione S-transferase (GST), maltose E binding protein, or protein A, respectively, to the target recombinant protein. Purified fusion proteins can be utilized in marker activity assays, e.g., direct assays or competitive assays described in detail below, or to generate antibodies specific for marker proteins for example.

Examples of suitable inducible non-fusion E. coli expression vectors include pTrc (Amann et al, (1988) Gene 69:301-315) and pET 1 Id (Studier et al., Gene Expression Technology: Methods in Enzymology 185, Academic Press, San Diego, Calif. (1990)60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gnl). This viral polymerase is supplied by host strains BL21(DE3) or HIS174(DE3) from a resident prophage harboring a T7 gnl gene under the transcriptional control of the la UV 5 promoter.

One strategy to maximize recombinant protein expression in E. coli beyond mutating the p15A locus, is to express the protein in a host bacteria with an impaired capacity to proteolytically cleave the recombinant protein (Gottesman, S., Gene Expression Technology: Methods in Enzymology 185, Academic Press, San Diego, Calif. (1990) 119-128). Another strategy is to alter the nucleic acid sequence of the nucleic acid to be inserted into an expression vector so that the individual codons for each amino acid are those preferentially utilized in E coli (Wada et al., (1992) Nucleic Acids Res. 20:2111-2118). Such alteration of nucleic acid sequences of the invention can be carried out by standard DNA synthesis techniques.

Another aspect of the invention pertains to host cells into which a nucleic acid molecule of the invention is introduced, e.g., enhanced plSA ori within a recombinant expression vector or a nucleic acid molecule of the invention containing sequences which allow it to homologously recombine into a specific site of the host cell's genome. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. Preferably, the host cell is a prokaryotic cell. For example, the invention can be expressed in bacterial cells such as E. coli. Other suitable host cells are known to those skilled in the art.

Vector DNA can be introduced into host cells via conventional transformation or transfection techniques. As used herein, the terms “transformation” and “transfection” are intended to refer to a variety of art-recognized techniques for introducing foreign nucleic acid, e.g., DNA, into a host cell, including calcium phosphate or calcium chloride co-precipitation, DEAE-dextran-mediated transfection, lipofection, or electroporation. Suitable methods for transforming or transfecting host cells can be found in Sambrook, et al (Molecular Cloning: A Laboratory Manual. 2nd, ed., Cold Spring Harbor Laboratory, Cold Spring Harbor Laboratory Press, Cold Spring Harbor, N.Y., 1989), and other laboratory manuals.

A host cell of the invention, such as a host cell in culture, can be used to produce, i.e., express, a recombinant protein. Accordingly, the invention further provides methods for producing a protein using the host cells of the invention. In one embodiment, the method comprises culturing the host cell of invention (into which a recombinant expression vector encoding a protein, or proteins, has been introduced) in a suitable medium such that a protein of the invention is produced. In another embodiment, the method further comprises recovering (e.g., isolating) the expressed protein from the culture medium or the host cell. Such recovery methods are well-known to those of skill in the art.

The features and other details of the invention will now be more particularly described and pointed out in the exemplification. It will be understood that the particular embodiments of the invention are shown by way of illustration and not as limitations of the invention. The principle features of this method are employed in various embodiments without departing from the scope of the invention.

Exemplification: Construction of pECI-122

In one embodiment of the present invention, a plasmid is constructed which is capable of protein expression in a vector with an enhanced p15A ori using plasmid pECI-077 as the starting vector backbone. The first part of this plasmid construction involves the addition of a constitutive promoter and once that is completed the second addition is the insertion of a gene into the multiple cloning site (MCS) for expression purposes. pECI-077 contains the enhanced p15A ori, described herein, the chloramphenicol acetyl transferase gene as a selectable marker, the rrnB transcriptional terminator, and a multiple cloning site. The MCS contains several restriction sites (e.g. NdeI, PstI, PvuI, SmaI, XhoI, Sac, and EcORI) NdeI is the first restriction endonuclease site for ease of cloning a target gene in frame. Plasmid pECI-077 was digested using BamHI and NdeI producing a 4.5 Kb linearized plasmid. The 4.5 Kb plasmid product was next subjected to agarose gel electrophoresis followed by gel purification and using Qbiogenes Geneclean II spin kit yielding a highly purified 4.5 Kb plasmid.

The alpha-amylase promoter region was prepared by PCR amplification. A forward primer was designed with a BamHI site

(SEQ ID NO. 18)

CGGGGATCCTTTATTTCGATTCACTCC;
a reverse primer

(SEQ ID NO. 19)

GGGAATTCCATATGAATGCCCTCCTTATAATCAAATGTTAC

was designed with an NdeI site. The alpha-amylase promoter (256 bp) was amplified from a previously characterized plasmid that contains the alpha-amylase gene and its promoter. A double digest of the 256 bp PCR DNA product was accomplished using BamHI and NdeI, which removed any overhanging ends of the forward and reverse primers. The digested, alpha-amylase promoter DNA was next subjected to agarose gel electrophoresis, followed by gel purification using Qbiogenes Geneclean II spin kit yielding a highly purified 256 bp DNA containing the alpha-amylase promoter.

The purified 4.5 Kb pECI-077 fragment was ligated to the purified 256 bp alpha-amylase promoter fragment to form a new plasmid pECI-084 (see FIG The ligated product was then transformed into BL21(DE3) cells and plated for transformants on-chloramphenicol containing plates. The plasmid pECI-077 has a nucleotide sequence that encodes for chloramphenicol resistance. Several transformants were obtained from the transformation. The transformed colonies were screened for the presence of the alpha-amylase promoter using the Polymerase Chain Reaction.

This new plasmid pECI-084 was then used to construct the final plasmid containing the gene that encodes the Acyl-CoA-binding Protein (ACBP). pECI-084 was digested with NdeI and XhoI yielding a 4.68 Kb DNA fragment. This fragment was then subjected to agarose gel electrophoresis and purified using Qbiogenes Geneclean II spin kit. Plasmid pECI-126 which contains the gene which encodes (ACBP) was digested with NdeI and XhoI and the digest was subjected to agarose gel electrophoresis and the 250 bp ACBP DNA fragment was gel purified using Qbiogenes Geneclean II spin kit.

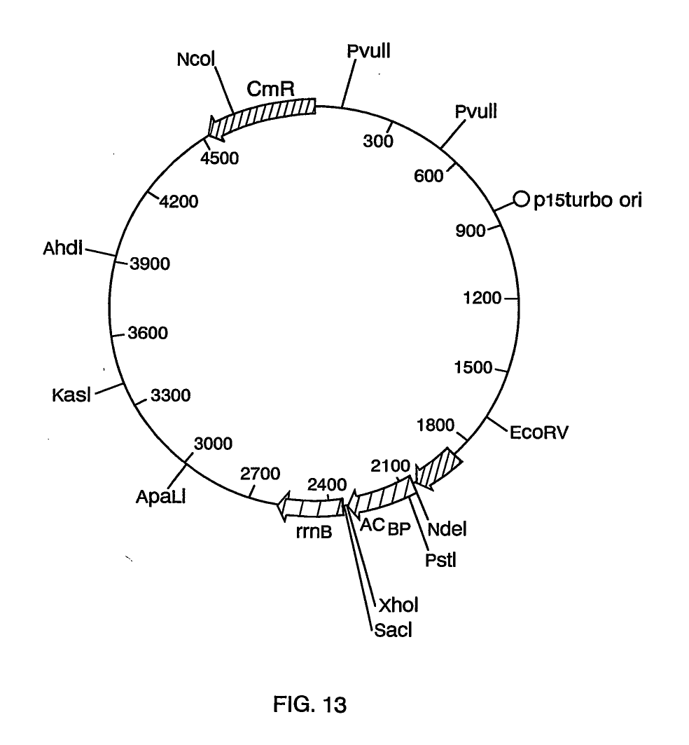

The 4.68 Kb pECI-084 fragment was then ligated to the 250 bp gel purified DNA fragment (from pECI-126). The ligated product was then transformed into BL21(DE3) cells and plated for transformants on chloramphenicol-containing plates to form a new plasmid, pECI-122 (see FIG. 13). The transformed colonies were then screened for the expression of the ACBP protein. Approximately 5 ml of 2×Circlegrow (CG) CM50 culture was prepared for 6 transformant colonies. The culture was permitted to grow overnight with agitation at approximately 37° C. The next day, the optical density (at A600 nm) of each culture was measured, and the cultures, were all normalized to a value of about 2.0 using 2×CG. About 1 ml of each culture was processed as follows in order to obtain ACBP protein. The samples were centrifuged in a microcentrifuge to pellet the cells. The supernatants were discarded and each pellet was resuspended in 100 ul of 2M Acetic acid and sonicated 3 by 5 seconds. The sonicated samples were then centrifuged for 15 minutes in a microfuge at maximum speed. The supernatants were then removed to a new tube and the following additions were made to each supernatant: 9 ul 1M Tris base, 100 ul 2× Laemmli sample buffer, and 35 ul of 2M Sodium hydroxide. The samples were then boiled for 5 minutes at 95° C.

A standard volume of each sample was analyzed by acrylamide gel electrophoresis. Coomassie blue staining of the gel was used to determine which, if any, transformant colonies were expressing the ACBP protein. The result of the gel indicated that transformant colonies 1,3 and 6 Were expressing the 10 Kd ACBP protein, with tranformants 3 and 6 yielding the greatest amount of protein. The expression level of ACBP seen with these 2 transformants (clones numbers 3 and 6) containing the constitutive alpha-amylase promoter were compared with the expression level of a plasmid containing the strong inducible T7 promoter. The results indicated that ACBP expression seen using the alpha-amylase promoter was comparable to that seen with the T7 promoter.

While this invention has been particularly shown and described with references to preferred embodiments thereof, it will be understood by those skilled in the art that various changes in form and details may be made therein without departing from the scope of the invention encompassed by the appended claims.

1. An origin of replication sequence comprising the nucleic acid sequence SEQ ID NO. 4, or a functional fragment thereof.

2. An origin of replication sequence comprising the nucleic acid sequence SEQ. ID. NO. 5, or a functional fragment thereof, wherein the sequence comprises a nucleotide mutation at one, or more nucleotide positions in the sequence, wherein the mutation or mutations result in enhanced function.

3. The origin of replication sequence of claim 2 wherein the mutations occur at one or more of the nucleotide positions selected from the group consisting of: 334, 441, 516, 657, 1032, 1188, 1326, 1354, 1359 and 1376.

4. The origin of replication sequence of claim 2 wherein the one or more mutations are selected from the group of mutations consisting of:
a) at position number 334 a thymidine is changed to an adenine;
b) at position number 411 a thymidine is changed to a cytosine;
c) at position number 516 an adenine is changed to a guanine;
d) at position 657 an adenine is changed to a guanine;
e) at position 1032 a thymidine is changed to a cytosine;
f) at position 1188 a thymidine is changed to an adenine;
g) at position 1326 an adenine is changed to a cytosine;
h) at position 1354 a thymidine is changed to a cytosine;
i) at position 1359 an adenine is changed to a guanine; and
j) at position 1376 an adenine is changed to a guanine.

5. A plasmid according to claim 4 wherein the origin of replication mutations comprise mutations e) to j).

6. A plasmid comprising an operatively linked origin of replication sequence comprising the nucleic acid sequence of SEQ ID NO. 4, or a functional fragment thereof.

7. The plasmid of claim 6 wherein the origin of replication sequence comprises a sequence that is at least 70% homologous to SEQ ID NO. 4.

8. The plasmid of claim 6 wherein the origin of replication sequence comprises a sequence that is at least 80% homologous to SEQ ID NO. 4.

9. The plasmid of claim 6 wherein the origin of replication sequence comprises a sequence that is at least 90% homologous to SEQ ID NO. 4.

10. A plasmid comprising an operatively linked origin of replication sequence of claim 1, wherein the plasmid is selected from the group consisting of: pECI-073, pECI-074, pECI-075, pECI-077, pECI-084 and pECI-122.

11. A cell containing a plasmid comprising the origin of replication according to claim 1.

12. A cell containing a plasmid according to claim 6.

13. A method of expressing one, or more exogenous proteins in a host cell, comprising:
a) transforming the host cell with a plasmid comprising:
i) an operatively linked origin of replication sequence according to any claim 1; and
ii) an operatively linked nucleic acid sequence encoding for one, or more exogenous proteins; and
b) maintaining the transformed host cell under culture conditions sufficient for expressing the protein or proteins in the cell.

14. The method of claim 13 further comprising:
c) recovering the expressed protein from the culture.

15. A method of expressing one, or more exogenous proteins in a host cell, comprising:
a) transforming the host cell with a plasmid according to claim 5, wherein the plasmid comprises an operatively linked nucleic acid sequence encoding for one, or more exogenous proteins; and
b) maintaining the host cell under culture conditions sufficient for expressing the protein or proteins in the cell.

16. The method of claim 15 further comprising:
c) recovering the expressed protein from the culture.

17. A method according to claim 13 wherein the plasmid comprises a plasmid selected from the group of: pECI-073, pECI-074, pECI-075, pECI-077, pECI-084 and pECI-122.

18. A cell containing a plasmid comprising the origin of replication according to claim 2.

19. A cell containing a plasmid comprising the origin of replication according to claim 3.

20. A cell containing a plasmid comprising the origin of replication according to claim 4.

21. A cell containing a plasmid comprising the origin of replication according to claim 5.

22. A cell containing a plasmid according to claim 7.

23. A cell containing a plasmid according to claim 8.

24. A cell containing a plasmid according to claim 9.

25. A method of expressing one, or more exogenous proteins in a host cell, comprising:
a) transforming the host cell with a plasmid comprising:
i) an operatively linked origin of replication sequence according to claim 2; and
ii) an operatively linked nucleic acid sequence encoding for one, or more exogenous proteins; and
b) maintaining the transformed host cell under culture conditions sufficient for expressing the protein or proteins in the cell.

26. A method of expressing one, or more exogenous proteins in a host cell, comprising:
a) transforming the host cell with a plasmid comprising:
i) an operatively linked origin of replication sequence according to claim 3; and
ii) an operatively linked nucleic acid sequence encoding for one, or more exogenous proteins; and
b) maintaining the transformed host cell under culture conditions sufficient for expressing the protein or proteins in the cell.

27. A method of expressing one, or more exogenous proteins in a host cell, comprising:
a) transforming the host cell with a plasmid comprising:
i) an operatively linked origin of replication sequence according to claim 4; and
ii) an operatively linked nucleic acid sequence encoding for one, or more exogenous proteins; and
b) maintaining the transformed host cell under culture conditions sufficient for expressing the protein or proteins in the cell.

28. A method of expressing one, or more exogenous proteins in a host cell, comprising:
a) transforming the host cell with a plasmid according to claim 6, wherein the plasmid comprises an operatively linked nucleic acid sequence encoding for one, or more exogenous proteins; and
b) maintaining the host cell under culture conditions sufficient for expressing the protein or proteins in the cell.

29. A method of expressing one, or more exogenous proteins in a host cell, comprising:
a) transforming the host cell with a plasmid according to claim 7, wherein the plasmid comprises an operatively linked nucleic acid sequence encoding for one, or more exogenous proteins; and
b) maintaining the host cell under culture conditions sufficient for expressing the protein or proteins in the cell.

30. A method of expressing one, or more exogenous proteins in a host cell, comprising:
a) transforming the host cell with a plasmid according to claim 8, wherein the plasmid comprises an operatively linked nucleic acid sequence encoding for one, or more exogenous proteins; and
b) maintaining the host cell under culture conditions sufficient for expressing the protein or proteins in the cell.

31. A method of expressing one, or more exogenous proteins in a host cell, comprising:
a) transforming the host cell with a plasmid according to claim 9, wherein the plasmid comprises an operatively linked nucleic acid sequence encoding for one, or more exogenous proteins; and
b) maintaining the host cell under culture conditions sufficient for expressing the protein or proteins in the cell.

32. A method according to claim 15 wherein the plasmid comprises a plasmid selected from the group of: pECI-073, pECI-074, pECI-075, pECI-077, pECI-084 and pECI-122.

33. A method according to claim 25 wherein the plasmid comprises a plasmid selected from the group of: pECI-073, pECI-074, pECI-075, pECI-077, pECI-084 and pECI-122.

34. A method according to claim 26 wherein the plasmid comprises a plasmid selected from the group of: pECI-073, pECI-074, pECI-075, pECI-077, pECI-084 and pECI-122.

35. A method according to claim 27 wherein the plasmid comprises a plasmid selected from the group of: pECI-073, pECI-074, pECI-075, pECI-077, pECI-084 and pECI-122.

36. A method according to claim 28 wherein the plasmid comprises a plasmid selected from the group of: pECI-073, pECI-074, pECI-075, pECI-077, pECI-084 and pECI-122.

37. A method according to claim 29 wherein the plasmid comprises a plasmid selected from the group of: pECI-073, pECI-074, pECI-075, pECI-077, pECI-084 and pECI-122.

38. A method according to claim 30 wherein the plasmid comprises a plasmid selected from the group of: pECI-073, pECI-074, pECI-075, pECI-077, pECI-084 and pECI-122.

39. A method according to claim 31 wherein the plasmid comprises a plasmid selected from the group of: pECI-073, pECI-074, pECI-075, pECI-077, pECI-084 and pECI-122.

40. The method of claim 25 further comprising:
c) recovering the expressed protein from the culture.

41. The method of claim 26 further comprising:
c) recovering the expressed protein from the culture.

42. The method of claim 27 further comprising:
c) recovering the expressed protein from the culture.

43. The method of claim 28 further comprising:
c) recovering the expressed protein from the culture.

44. The method of claim 29 further comprising:
c) recovering the expressed protein from the culture.

45. The method of claim 30 further comprising:
c) recovering the expressed protein from the culture.

46. The method of claim 31 further comprising:
c) recovering the expressed protein from the culture.
